(19) United States
(12) Patent Application Publication
(10) Pub. No.: US 2024/0076682 A1
(43) Pub. Date: Mar. 7, 2024

(54) PROMOTER OF THE TRANSCRIPTION OF NUCLEIC ACID SEQUENCE AN ITS USE

(71) Applicant: UNIVERSITA' DEGLI STUDI DI BARI ALDO MORO, Bari (IT)

(72) Inventors: René Massimiliano MARSANO, BARI (IT); Antonio PALAZZO, BARI (IT)

(21) Appl. No.: 17/897,857

(22) Filed: Aug. 29, 2022

(51) Int. Cl. C12N 15/66; C12N 15/11

(52) U.S. Cl. CPC C12N 15/66; C12N 15/11; C12N 15/111; C12N 2800/10; C12N 2800/90

(57) ABSTRACT

The present invention relates to the field of biotechnologies and in particular to a nucleotide sequence having promoter activity of the transcription in prokaryotic and eukaryotic cell systems.

Sequence listing 223006.xml, created on Mar. 29, 2023 and of size of 4585 bytes is incorporated herein by reference.

### STATE OF THE ART

The recombinant DNA technology allows the ectopic expression of nucleic acid sequences of interest, preferably gene sequences coding for a protein product, in host cell systems which normally do not express said gene sequences. This allows obtaining high levels of the protein of interest for the therapeutic, industrial or research use.

The ectopic expression, or else the forced expression of one or more nucleic acid sequences of interest in cell systems which normally do not express said nucleic acid sequences, is done by inserting at least one nucleic acid sequence of interest, for example at least one gene sequence, in an expression vector which then will be introduced into the selected host cell system.

The techniques of ectopic expression of gene sequences currently available and used are based on the forced transcription of sequences of interest into cell types that do not normally express such genes or at times in cell development or differentiation that do not involve the expression of that gene.

In general, the purposes for which ectopic expression studies are carried out range from characterizing the functions of as yet unknown gene sequences to producing recombinant proteins in heterologous systems such as, for example, producing therapeutically useful proteins (e.g., insulin) in bacteria. The ectopic expression in the bacteria remains, in fact, the best option for producing high-level, low-production-cost proteins. However, the ectopic expression in eukaryotic systems remains the first choice because the post-translational modifications that allow the ectopically expressed proteins to retain their biological activity are ensured in these systems.

The known host cell systems are, therefore, both prokaryotic in origin, usually bacteria, and eukaryotic in origin, more complex cell systems, for example, animals or plants. For example, the choice of the host cell system may depend on the type of protein intended to be expressed. As mentioned above, the ectopic expression in the bacteria is the best option for producing high quantities of proteins at low production costs. However, eukaryotic systems need to be used, for example, when the protein requires post-translational modifications to keep its biological activity.

The expression vector is a nucleic acid molecule able to transport and drive the expression of DNA sequences of interest inside a host cell system.

It is imperative that the expression vector contains all the molecular signals needed to the host cell system to transcribe the gene sequence of interest.

Among the molecular signals the expression vector must contain, a key element is represented by the promoter.

The promoter is a nucleotide sequence which is upstream of the sequence coding for the gene of interest and is recognized by the RNA polymerase of the host cell system, which allows to initiate the transcription of sequences placed immediately downstream of it. The transcriptional promoter is a cis-acting factor, and it overlaps with the initiation site of the transcription.

From the functional point of view, the promoter performs the same function both in the eukaryotic genomes and in the prokaryotic (bacterial) genomes but, from the structural point of view and the organization of the recognition sequences, the prokaryotic and eukaryotic promoters are different.

The prokaryotic (e.g., bacterial) promoter has a very simple structure and consists of at least three sequences for the recognition by the RNA polymerase, the −10, −35 elements and the upstream (UP) element. The −10 and −35 elements are recognized by the ζ subunit of the RNA polymerase, while the UP element is recognized by the a subunit.

The eukaryotic promoter also has a modular structure in which different elements, such as the CAAT box, the TATA box and the GC box, may be recognized. The CAAT box is generally located at position −80 relative to the initiation site of the transcription and is the module which confers specificity and strength to the promoter, while the TATA box is responsible for the formation of the transcription initiation complex. In fact, for a eukaryotic promoter to function properly, it must be able to assemble the transcription initiation complex, whose main catalytic activity is enacted by the RNA polymerase. There are three different eukaryotic RNA polymerases of which the RNA polymerase II is responsible for the transcription of most genes. The transcription factors confer specificity to the eukaryotic promoters even at the level of different tissue types in the same organism.

At present the problem of the selection of the cell system requires that many different expression vectors must be used and “designed” ad hoc based on the cell system in which they will be used.

To date, few sequences capable of functioning as promoters in different cell systems are known for the biology underlying the gene transcription. This is because each cell has its own combination of transcription factors that recognize specific functional domains present on the promoter, the interaction of the right signals on the appropriate domains triggers the activation of the transcriptional system that eventually leads to the production of messenger RNA.

US20210180052A1 and EP2772539B1 describe nucleotide sequences having promoter-function and capable of inducing the expression of genes of interest in different prokaryotic and eukaryotic cell systems.

However, the known promoters, although capable of promoting the gene expression in various cell systems, show limitations in their use. For example, the known promoters may exhibit excessively high activity, such that a production of heterologous proteins may exert a toxic effect on the host cell and/or, in the case of eukaryotic cells, such that cell development and differentiation are adversely affected.

Therefore, there is particular need of providing sequences having promoter function capable to activate the expression of the gene downstream of them, both in prokaryotic cells and in eukaryotic cells. Furthermore, there is a need to provide sequences having promoter function and expression vectors that include them, that allow expression levels of genes in different cell systems, both prokaryotic and eukaryotic, safely and without deleterious effects on the host cells.

### OBJECTS OF THE INVENTION

It is a purpose of the present invention to provide a sequence having promoter activity of the transcription of a nucleic acid sequence of interest, for example a gene, in prokaryotic cell systems and eukaryotic cell systems.

It is also a purpose of the present invention to provide an expression vector which allows obtaining expression levels of a nucleic acid sequence of interest such that said vector may be used safely and without deleterious effects on the host cells.

It is another purpose of the invention to provide a method for expressing a sequence of interest in a prokaryotic and eukaryotic host cell system.

### BRIEF DESCRIPTION OF THE FIGURES

FIG. 1 is a schematic representation of the organization of the transposable element PiggyBac of Trichoplusia ni from which the sequence of the invention has been derived. The structure of the two terminal sequences with the inverted repeats of 13 and 19 base pairs is depicted. The 5′-end terminal sequence corresponds to the promoter.

FIG. 2 shows schematically an embodiment of the expression vector of the invention.

FIG. 3 depicts the results of a bioluminescence assay, which are suitable for measuring the activity of the sequence of the invention at the episomal level, which bioluminescence assay is carried out in four eukaryotic and prokaryotic cell systems.

FIG. 4 depicts a scheme of using the sequence of the invention in selecting clones expressing the gene for blasticidin resistance in stable form in cultured human cells and in episomal form in bacterial cells.

### DESCRIPTION OF THE INVENTION

The aforementioned purposes are achieved by the object of the present invention, i.e. an isolated nucleic acid sequence having promoter activity of the transcription in prokaryotic and/or eukaryotic cell systems, comprising or having the sequence CCCTAGAAAGATAGTCTGCGTAAAATTGACGCATGCATTCTTGAAATATTGC TCTCTCTTTCTAAATAGCGCGAATCCGTCGCTGTGCATTTAGGACATCTCAG TCGCCGCTTGGAGCTCCCGTGAGGCGTGCTTGTCAATGCGGTAAGTGTCACT GATTTTGAACTATAACGACCGCGTGAGTCAAAATGACGCATGATTATCTTTT ACGTGACTTTTAAGATTTAACTCATACGATAATTATATTGTTATTTCATGTTC TACTTACGTGATAACTTATTATATATATATTTTCTTGTTATAGATATCGTGAC TAATATATAATAAA (SEQ. ID. NO. 1), or a sequence having at least 80% sequence identity with said sequence SEQ ID NO. 1.

According to the invention, said isolated sequence is a promoter of the transcription in prokaryotic and/or eukaryotic cell systems, which comprises the sequence CCCTAGAAAGATAGTCTGCGTAAAATTGACGCATGCATTCTTGAAATATTGC TCTCTCTTTCTAAATAGCGCGAATCCGTCGCTGTGCATTTAGGACATCTCAG TCGCCGCTTGGAGCTCCCGTGAGGCGTGCTTGTCAATGCGGTAAGTGTCACT GATTTTGAACTATAACGACCGCGTGAGTCAAAATGACGCATGATTATCTTTT ACGTGACTTTTAAGATTTAACTCATACGATAATTATATTGTTATTTCATGTTC TACTTACGTGATAACTTATTATATATATATTTTCTTGTTATAGATATCGTGAC TAATATATAATAAA (SEQ. ID. NO.1) or a sequence having at least 80% sequence identity with said sequence SEQ ID NO. 1.

In other words, according to the present invention, the expression “isolated nucleic acid sequence having activity of transcription promoter” and the expression “transcription promoter” or, more simply “promoter” are equivalent and may be used alternatively.

For example, according to the present invention, sequences may be used having at least 85%, preferably at least 90%, more preferably at least 95% sequence identity with the sequence SEQ: ID NO. 1.

Preferably, the isolated sequence of the invention is the sequence SEQ. ID NO. 1.

The transcription is the transfer process of the genetic information from the DNA to the RNA which then will be translated into the protein.

The nucleotide sequence SEQ. ID. NO. 1 of the invention having promoter activity of the transcription in prokaryotic and eukaryotic cell systems was arbitrarily called “PB-XpreS” (an English acronym for “PB-Expression System”).

Thus, within the present description we may refer to the nucleotide sequence of the invention having transcription activator activity in prokaryotic and eukaryotic cell systems also with the acronym “PB-XpreS” or “PB-XpreS promoter sequence”.

The PB-XpreS promoter sequence of the invention has sequence SEQ. ID. NO. 1:

CCCTAGAAAGATAGTCTGCGTAAAATTGACGCATGCATTCTTGAAAT
ATTGCTCTCTCTTTCTAAATAGCGCGAATCCGTCGCTGTGCATTTAGGA
CATCTCAGTCGCCGCTTGGAGCTCCCGTGAGGCGTGCTTGTCAATGCGG
TAAGTGTCACTGATTTTGAACTATAACGACCGCGTGAGTCAAAATGACG
CATGATTATCTTTTACGTGACTTTTAAGATTTAACTCATACGATAATTA
TATTGTTATTTCATGTTCTACTTACGTGATAACTTATTATATATATATT
TTCTTGTTATAGATATCGTGACTAATATATAATAAA

The sequence SEQ. ID. NO. 1, identified in the 5′ region of the PiggyBac transposon (PB) of Trichoplusia ni, is constituted by 328 bp (“base pairs”). It has been surprisingly observed that the sequence of the invention has the ability to promote the gene transcription in both prokaryotic and eukaryotic cell systems, even without the need to use genetically engineered host systems for the purpose of enabling the transcription of exogenous genes.

The nucleotide sequence according to the present invention is thus particularly suitable and useful for both basic research studies, testing and industrial production of recombinant proteins, toxicological studies, and novel and emerging trans-kingdom gene therapy.

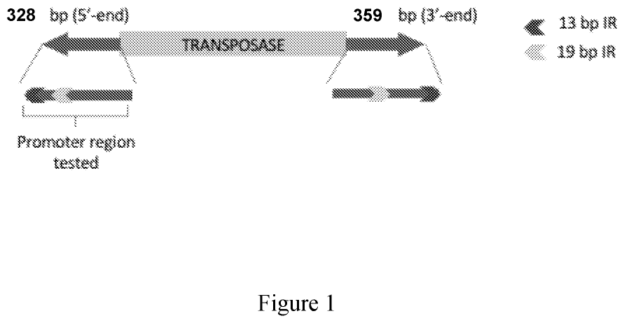

The sequence according to the invention comprises the activating sequence of the transcription of a transposon, a mobile genetic element widely known, per se, in the literature for several decades, namely the PiggyBac element schematized in FIG. 1.

FIG. 1 is a schematic representation of the PiggyBac transposon organization. The transposase gene is flanked by the 5′-end and 3′-end terminal regions. Both terminal regions include two terminal sequences with inverted repeats of 13 and 19 base pairs. The PiggyBac transposon comprises the sequence of the invention, which corresponds to the 5′-end portion.

The transposons, also named transposable elements, are nucleic acid sequences in prokaryotic and eukaryotic genomes and capable of changing map positions in the genome.

In the transposable elements such as the one schematized in FIG. 1, the terminal sequences (5′-end and 3′-end) are essential for the transposition because they contain the recognition sites for transposase, the enzyme which catalyzes the excision and integration reactions of the transposable element. Furthermore, for the transcription of the gene coding for transposase to occur, a transcription activator sequence, i.e., the actual promoter, must be contained in the 5′ terminal sequence.

In embodiments, the sequence of the invention comprises the 5′-end of a PiggyBac transposable element, preferably of Trichoplusia ni, said 5′-end preferably having a length of 328 bp and preferably comprising IR sequences of 13 bp or 19 bp.

In embodiments, the sequence of the invention is the 5′-end nucleotide sequence of a PiggyBac transposable element of Trichoplusia ni. In embodiments, the sequence of the invention is a nucleic acid sequence having at least 80 percent sequence identity, preferably at least 90 percent, even more preferably at least 95 percent with the 5′-end nucleotide sequence of a PiggyBac transposable element of Trichoplusia ni.

Advantageously, the sequence of the invention may be used for the ectopic expression of nucleic acid segments of interest in prokaryotic and eukaryotic cell systems.

According to the invention, the PB-XpreS promoter sequence is between the first nucleotide and the nucleotide preceding the first ATG codon of the transposase gene inside the PiggyBac transposable element of Trichoplusia ni. The ATG codon (triplet AUG on the mRNA) is the specific sequence having 3 nucleotides (triplet) which is used as starting codon of the translation of the mRNA in protein. ATG codon codes for methionine.

Therefore, methionine is the amino acid which occupies the N-terminal of all the proteins of the eukaryotes and archaeobacteria.

According to the invention, the sequence of the invention may be used for the activation of the transcription of at least one nucleic acid sequence of interest in a number of different host cell systems, for example in prokaryotic host cells and/or eukaryotic host cells. By “host cell system” is meant herein to denote the cell (or cells) in which nucleic acid sequences of interest, for example gene sequences, are introduced by expression vectors.

Surprisingly, it was indeed observed that the sequence of the invention activates the transcription of reporter genes in cell systems very different from each other as the eukaryotic and prokaryotic ones. In particular, a significant ability to actively transcribe was observed in systems such as mammalian (human), insect and yeast cells, as well as in bacterial systems.

Therefore, according to the invention, the sequence of the invention may be used for the activation of the transcription of at least one nucleic acid sequence of interest in prokaryotic cells, such as for example bacteria, and/or in eukaryotic cells. In other words, an object of the present invention is the use of an isolated sequence according to the invention as a promoter of the transcription in prokaryotic and/or eukaryotic cell systems.

In embodiments, the prokaryotic cells are selected from bacterial cells, preferably from bacterial cells of the Escherichia coli species.

In embodiments, the eukaryotic cells are selected from mammalian cells, reptile cells, amphibian cells, bird cells, insect cells and yeast cells.

According to the invention, the sequence of the invention is used for the activation of the transcription of at least one nucleic acid sequence of interest when inserted into an expression vector, said expression vector being then introduced into host cell systems. According to an embodiment, the sequence of the invention is used for the activation of the transcription of nucleic acid sequences of interest non-coding for proteins. In fact, it is known that some non-coding genome regions are responsible for producing regulatory RNAs of the gene expression, such as for example miRNAs. Said miRNAs are endogenous molecules of non-coding RNAs active in the regulation of the gene expression at the transcriptional and post-transcriptional level.

According to the invention, the sequence of the invention is placed upstream of nucleic acid sequences of interest.

Therefore, in an embodiment, the sequence of the invention may be placed upstream of nucleic acid non-coding sequences, in order, for example, to study the function of new elements of the genome in the basic research.

According to a particularly preferred embodiment, the sequence of the invention is used for the activation of the transcription of at least one sequence coding for a protein of interest, for example one gene sequence. According to said particularly preferred embodiment, the sequence of the invention is placed upstream of said gene sequences coding for proteins of interest. For example, sequences of interest are sequences coding for a protein product of interest and comprise the exons of the gene that codes for such protein.

The exon is the gene (eukaryotic or of archaeobacteria) part which is transcripted in RNA, together with the introns. Subsequently, by a process defined “splicing”, the introns are removed, whereas the exons are linked in the mature RNAs and translated into an amino acid sequence.

It has been observed that the sequence of the invention has a promoter activity which is defined “weak”. A weak promoter is a promoter able to moderately activate the expression of the sequence of interest placed downstream of it, such as for example a gene, with respect to promoters having intense transcriptional activity.

The “strength” of a promoter is determined by comparing the ability to activate the transcription of the promoter under consideration with the ability to activate the transcription of a reference, so-called “strong” promoter. There are promoters that are traditionally thought to be strong that are referred to when studying new ones. For example, the “CAT,” “URA,” and “copy” promoters are considered strong, whereas the promoter of “SV40” is considered having medium strength.

Therefore, said sequence of the invention is particularly suitable to be used for the expression of proteins of which a medium-low level is desired.

The expression level achievable through a promoter under consideration may be determined by comparison with the expression level of genes expressed using known promoters.

The gene expression level may be measured by techniques known, per se, in the art such as RT-qPCR. Advantageously, the sequence of the invention is particularly suitable to be used for the expression of proteins which, if they are expressed in high amounts, would be toxic for the host cell.

Therefore, the promoter sequence of the invention may be used as weak promoter for the expression of coding sequences or non-coding sequences both in prokaryotic and eukaryotic cell systems. For example, the sequence of the invention may be used as weak promoter in prokaryotic cell systems, such as bacteria, and in eukaryotic cell systems, such as mammalian cells.

In embodiments, the nucleotide sequence of interest is a reporter gene or selection marker sequence.

By the term “reporter gene” is meant a gene whose activity is easily monitored by histochemical or immunological assays, and which codes for a gene product that may be used to study the activity of regulatory sequences of another gene of interest.

Examples of reporter genes are the gene coding for GFP (green fluorescent protein) and the gene expressing luciferase (Luc).

By the term “selection marker” or “gene marker” is meant to denote sequences that code for products that may be used as morphological, biochemical or genetic markers associated with specific traits of the organism into which they are inserted. Such selection markers allow the selection of organisms carrying certain traits.

Therefore, the sequence of the invention may be used for producing selection cassettes, which express marker genes capable of making selectable eukaryotic and prokaryotic cells into which the vector has been introduced.

Examples of selection marker genes are GFP (green fluorescent protein), genes expressing antibiotic resistance proteins (e.g., bla and cat genes), the gene expressing luciferase (Luc) and genes expressing toxin/antitoxin systems (e.g., ccdA/ccdB genes).

The sequence of the invention is suitable to be used for the preparation of expression vectors.

Therefore, another object of the present invention is an expression vector comprising the isolated sequence of the invention, preferably comprising the SEQ sequence. ID. NO. 1.

According to the invention, an expression vector is a nucleic acid molecule able to carry a nucleic acid sequence in a prokaryotic and/or eukaryotic host cell and able to obtain the expression of said nucleic acid sequence in said host cells.

The expression vector comprising the sequence of the invention, particularly comprising the sequence SEQ. ID NO. 1, was arbitrarily named “pPB-XpreS”. Thus, within the present description we may refer to the expression vector comprising the sequence of the invention also with the acronym “pPB-XpreS”.

According to one of its aspects, the expression vector of the invention comprises the sequence of the invention, preferably the sequence SEQ: ID NO. 1, upstream of a nucleic acid sequence of interest. According to the invention, the expression vector further comprises at least one selection marker, such as for example at least one antibiotic resistance gene, and an origin of bacterial replication.

According to a particularly preferred embodiment, the expression vector comprises the sequence of the invention upstream of a nucleic acid sequence of interest, said nucleic acid sequence of interest being a gene of interest (transgene). By the term expression vector is meant herein to refer to any nucleic acid molecule for cloning and transferring a nucleic acid into a host cell. By the term “cloning” is meant herein to denote inserting DNA sequences into expression vectors able to transcribe the sequence (for example, a gene) of interest inside the host cell into which they are inserted.

For example, expression vectors according to the invention are considered the plasmids, cosmids, phages, bacterial artificial chromosomes (BAC), yeast artificial chromosomes (YAC) and viral vectors.

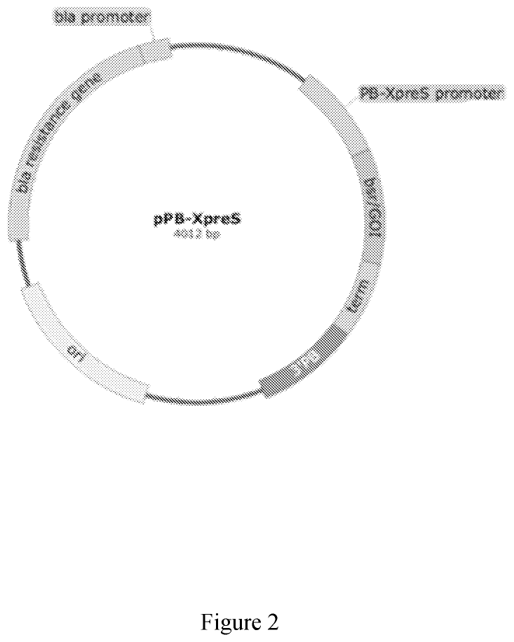

In FIG. 2 an embodiment of the expression vector according to the invention is depicted.

The expression vector depicted in FIG. 2 is a plasmid vector, that is, a plasmid, comprising the PB-XpreS promoter sequence of the invention. The PB-XpreS promoter enables the activation of the transcription of the gene of interest and a resistance gene to the blasticidin antibiotic. The resistance gene to the blasticidin antibiotic, which may be replaced by the gene of interest, are shown together, depicted by the name “bsr/GOI,” in FIG. 2. Downstream of the resistance gene to blasticidin and of the gene of interest, there is a transcription terminator (depicted as “term” in FIG. 2) and the 3′-end region of the PiggyBac transposon (depicted as “3′PB” in FIG. 2). In the vector depicted in FIG. 2, a bacterial replication origin (depicted in FIG. 2 as “ori”), a resistance gene to the beta-lactam antibiotics (depicted in FIG. 2 as “bla resistance gene”) and its promoter (depicted as “bla promoter”) are also schematized.

The expression vector depicted in FIG. 2 consists of 4012 base pairs (bp).

The expression vectors according to the present invention may be prepared according to methods known, per se, in the art.

The expression vectors are used to introduce a specific gene into target cells and may carry out the process of gene transcription to which the protein synthesis then follows to produce the protein encoded by the gene. The expression vectors are, in general, basic tools in biotechnology for the production of proteins.

In embodiments, the expression vector comprises at least one nucleic acid sequence of interest downstream of the sequence of the invention, for example downstream of the 5′ terminal sequence of the PiggyBac transposon. In embodiments, the nucleic acid sequence of interest is upstream of the 3′ terminal sequence of the PiggyBac transposon.

The expression vectors according to the present invention advantageously enable the transcription of nucleic acid sequences of interest, e.g., genes, both in prokaryotic and eukaryotic cells without the need to use host systems that are genetically engineered specifically to make such host systems suitable for the expression of exogenous nucleotide sequences.

Furthermore, advantageously, the expression vectors according to the invention may easily be used for the transfection techniques of any cell line, for example for cultured cell lines for basic research studies on the gene expression, for producing recombinant proteins or toxicological and pharmacological studies.

By “transfection” is meant herein to denote the introduction process of exogenous biological material into eukaryotic cells, in most cases mammalian cells. The transfection process may be carried out in vitro on target cells in long-term cell cultures, ex vivo on cells isolated from an organism and transferred on culture medium and in vivo directly on cells of an organism.

Furthermore, the expression vectors according to the invention may easily be used for the bacterial transformation and bacterial transduction techniques.

By “transformation” is meant herein to denote a molecular biology technique used to introduce genetic material into bacteria cells.

By “bacterial transduction” is meant to denote the passage of the DNA of a bacterium to another bacterium by a phage. A phage, also called bacteriophage, is a virus able to infect the bacterial cells. This allows inserting, in the bacterial genome, a sequence of interest present in the phage, for example a gene sequence.

Advantageously, the sequence according to the invention has the ability to activate the nucleotide sequence transcription even when it is integrated on host chromosomes.

According to an embodiment, the expression vectors of the invention are used for the gene therapy.

By “gene therapy” is meant herein to denote inserting, inside specific host cells, expression vectors comprising specific gene sequences of interest in order to cure diseases, preferably genetic diseases.

Advantageously, given its non-viral origin, the sequence of the invention exhibits reduced immunogenicity and cytotoxicity and may be safely used for the development of vectors that may be used for gene therapy.

Therefore, the expression vectors comprising the sequence of the invention for the use in gene therapy comprise at least one gene sequence of interest downstream of the sequence of the invention.

In an embodiment, the expression vectors of the invention may be used for the trans-kingdom gene therapy. The trans-kingdom gene therapy allows transferring therapeutic material, in the form of nucleic acids and proteins, to mammalian cells by using cells belonging to different Kingdoms (for example bacteria and fungi) and by the use of expression vectors for producing the therapeutic material inside target cells transfected with the vector of the invention.

Therefore, by “therapeutic material” is meant herein to denote nucleic acids of interest and proteins of interest introduced into mammalian cells in order to cure diseases.

Therefore, object of the present invention is an expression vector comprising the promoter of the invention, for its use in gene therapy, in particular, in the trans-kingdom gene therapy.

Still an object of the invention is a method for performing the gene therapy with the expression vector according to the invention, comprising the step of inserting, into specific host cells, said expression vector for the purpose of curing genetic diseases.

Further object of the invention is a method for expressing at least one nucleic acid sequence of interest in a prokaryotic and/or eukaryotic host cell which comprises the steps of:
a) cloning said at least one nucleic acid sequence of interest into an expression vector comprising the sequence of the invention, preferably the sequence SEQ. ID. NO. 1;
b) introducing said expression vector into the host cell;
c) culturing said prokaryotic and/or eukaryotic host cell by applying suitable conditions to obtain the expression of said nucleic acid sequence of interest.

According to a preferred embodiment, the expression vector comprises the sequence of the invention upstream of a transgene gene sequence.

According to the invention, cloning the DNA sequence of interest inside an expression vector in step a) is carried out by techniques known, per se, in the art.

Techniques suitable for cloning the DNA sequence of interest inside an expression vector are, for example, cloning by using restriction endonucleases, cloning by using recombinases, and cloning by using Gibson Assembly®.

According to the invention, step b) of introducing the expression vector into the host cell may be carried out by transformation or transfection.

Techniques suitable for performing the transformation of prokaryotic cells or the transfection of eukaryotic cells are known, per se, in the art.

For example, the transformation into prokaryotic cells may be performed by using heat shock method on cells made competent with calcium chloride or rubidium chloride, while the transfection into eukaryotic cells may be performed using lipofection agents, or with poly-cations, or by nucleofection.

Step c) of cultivating the host cell under suitable conditions depends on the type of cell, for example prokaryotic cell or eukaryotic cell, and on specific cell sub-type, e.g. kidney cell and neuron. The culture conditions of different specific cell sub-types are known in the art and, therefore, step c) of the method according to the invention may be carried out by using known culture conditions for each different type of host cell.

The present invention has many advantages over what is known in the art.

The sequence of the invention enables the development of multi-platform expression vectors, that is, capable of expressing exogenous nucleotide sequences both in prokaryotic cells and eukaryotic cells, particularly in bacterial, human and insect cells.

Advantageously, the expression vector of the invention is a valid alternative to expression vectors currently available. In fact, the sequence of the invention and the expression vectors comprising it allow to quickly switch from experiments and assays in a cell system to another of different origin. It follows that the chosen procedures of the final platform for the expression of a sequence of interest, such as for example a transgene (and related recombinant protein) will be greatly accelerated.

Even more advantageously, the present invention enables the expression of genes or sequences of interest in parallel after a single cloning operation. In other words, a unique vector comprising the nucleotide sequence of interest will be able to be inserted into different cell systems to carry out parallel experiments. Therefore, the sequence of the invention and the expression vector comprising said sequence allow reducing the research costs since it will no longer be necessary to build or buy different known expression vectors for each different type of cell to be used in the experimental analysis.

Further advantage of the present invention is that the sequence of the invention exhibits lower transcriptional promoter activity than that exhibited by known and commercialized promoters. This makes the sequence of the invention particularly suitable for use in the study of gene expression modifiers that act directly on the promoters, and for the heterologous expression of proteins that, in high amounts, are found to be toxic to the host cell.

For example, the expression vector may be used for the study of modifiers of the gene expression acting directly on the promoters, such as for example of the repressors or activators of transcription. In fact, by using the expression vector of the invention, comprising the sequence of the invention, having weak promoter activity, the effect of the expression modifiers, for example having repression effects of the transcription, is immediately evident by the drastic drop of the level of protein produced. By using the expression vector of the invention, the effect of the expression modifiers having activation effects of the transcription, is immediately evident by the very large amount of protein produced.

Furthermore, the ability to induce transcriptional activity in a more restrained way than the canonical promoters known as “strong” promoters has the advantage of making the sequence of the invention particularly suitable in the use for the expression of effector proteins of Cas-type protein-based genome editing systems (CRISPR associated sequences). In fact, it has been shown that constant high expression of these proteins erodes the specificity of the system and has negative influences on the cell development and differentiation.

The sequence of the invention allows instead the expression of such proteins in a highly specific way free of negative influences on the cell development and differentiation.

A further advantage is that the sequence of the invention turns out to be particularly suitable for the use in gene therapy, compared with promoters known in the art.

Another advantageous aspect of the present invention is that the sequence of the present invention may be conjugated to the integration system based on the PiggyBac transposable element. In eukaryotic cells, plasmids are soon lost, since eukaryotic cells lack the structures to replicate the plasmids and pass them on to daughter cells. By conjugating the promoter of the invention to the PiggyBac transposon, the expression system may be transformed from a transient system into a stable system, as the PiggyBac transposon is capable of integrating into the chromosomes, with the advantage of being stably maintained over time in the cells.

Furthermore, the sequence of the present invention allows for significantly higher transcriptional performance and versatility, relative to the cell system used, than the promoters known in the art.

Through systematic analysis of promoters isolated from transposable elements and by applying functional assays, the Applicant was able to observe the properties of the sequence according to the invention and its potential applications, particularly in the biotechnology field.

The present invention will now be illustrated through the following experimental section, which is to be considered purely illustrative and not limiting.

### EXPERIMENTAL SECTION

### Example 1: Construction of an Expression Vector Comprising the Sequence of the Invention

La Sequence of the Invention PB-XpreS

CCCTAGAAAGATAGTCTGCGTAAAATTGACGCATGCATTCTTGAAAT ATTGCTCTCTCTTTCTAAATAGCGCGAATCCGTCGCTGTGCATTTAGGACAT CTCAGTCGCCGCTTGGAGCTCCCGTGAGGCGTGCTTGTCAATGCGGTAAGTG TCACTGATTTTGAACTATAACGACCGCGTGAGTCAAAATGACGCATGATTAT CTTTTACGTGACTTTTAAGATTTAACTCATACGATAATTATATTGTTATTTCA TGTTCTACTTACGTGATAACTTATTATATATATATTTTCTTGTTATAGATATC GTGACTAATATATAATAAA (SEQ ID. NO. 1) has been cloned into the plasmid expression vector pGL3B-Basic (Promega Corporation) upstream of the gene sequence of luciferase. Such pGL3-Basic vector contains a reporter gene coding for the luciferase enzyme of the Photinus pyrahs firefly (luc gene).

Luciferase is an enzyme used for the quantitative analysis of elements that potentially regulate the gene expression both in cis and trans.

The plasmid has been cut with restriction enzymes XhoI and NcoI so to be linearized.

The PB-XpreS promoter sequence of the invention has been amplified by primers having at their ends the sequences recognized by the restriction enzymes XhoI and NcoI.

The amplification of the PB-XpreS promoter sequence was obtained by using the PB-TAC-ERN plasmid clone (Addgene Plasmid #80475) as a template and by using the primers:

PB-XpreS_fw primer(forward):
(SEQ. ID. NO. 2)
atcgCTCGAGccctagaaagatagtctgcgta
PB-XpreS_rev primer(reverse):
(SEQ. ID NO. 3)
atcgCCATGGtttattatatattagtcacgat

The PCR reactions to obtain the amplified PB-XpreS promoter sequence have been carried out in standard conditions as mentioned.

Thermal Cycling Parameters
1 cycle:
Denaturation for 3′ at 94° C.
35 cycles:
Denaturation for 30″ at 94° C.
Annealing for 30″ at 51° C.
Elongation for 20″ at 72° C.
1 cycle:
7′ at 72° C.
4° C. ∞

The reaction mixture for the amplification of the PB-XpreS promoter sequence is constituted as follows:
Reaction buffer 1×
1.5 mM Mg²⁺
0.2 mM dNTP mix
0.4 μM PB-XpreS_fw primer
0.4 μM PB-Xpress_rev primer
1 U Platinum Taq
1-5 ng Template DNA
Water up to 50 μl

The enzyme used is Platinum Taq polymerase (Invitrogen, Life Technologies).

The PB-XpreS_fw (forward) (SEQ. ID. NO. 2) and PB-XpreS rev (reverse) primer oligonucleotide (SEQ. ID. NO. 3) contain target sequences for the restriction enzyme XhoI and NcoI (depicted in uppercase in the primer sequences). The amplification fragments obtained have been cloned in the Xhol and Ncol sites of the pGL3-Basic plasmid vector (PROMEGA).

The pGL3-Basic vector is particularly useful with the purpose of measuring the activity of promoters thanks to the presence of a synthetic polyadenylation sequence in the multiple cloning site, which reduces the transcriptional background due to non-specific sequences upstream of the test sequence.

The enzymatic digestion of the amplified PB-XpreS promoter sequence and pGL3-B vector has been carried out with the following method.

Digestion Reaction for the PCR Samples

PB-XpreS Amplification 1 μg
Buffer H (NEB) 1×
2U Enzyme NcoI (NEB)
2U Enzyme XhoI (NEB)
Water up to 25 μl

The reactions have been carried out for 2 h at 37° C.

Digestion Reaction for the pGL3-B Plasmid
pGL3-B DNA 1 μg
Buffer H NEB 10×1×
2U Enzyme NcoI NEB
2U Enzyme XhoI NEB
Water up to 25 μl

The reactions have been carried out for 2 h at 37° C.

Ligation of the Digested Fragments
T4 DNA Ligase Reaction Buffer 1×
digested pGL3B 30 ng
PB-XpreS sequence (SEQ. ID NO. 1) 10 ng
1U T4 DNA ligase (NEB)
Water up to 20 μl

Reactions carried out at 15° C. for about 16 hours.

The resulting vector, according to the invention, is referred to as pPB-XpreS.

The recombinant plasmid clones have been purified with commercial kits (QIAGEN plasmid mini kit), quantified by measurement at Nanodrop and subsequently aliquots of 1 μg of each single plasmid have been carried into the cells of interest.

### Example 2: Luciferase Assay for Defining the Transcription Promoter Activity of the Sequence of the Invention and Transposition Assays

To demonstrate the efficiency of the PB-XpreS sequence in the transcription of the luciferase gene, a bioluminescence assay, that is, an assay of luciferase enzyme expression has been set up by using circular molecules of DNA (episomes).

The expression vector as obtained in Example 1, which comprises the sequence SEQ. ID NO. 1 according to the invention, was carried into the cells of interest (1 μg aliquots).

In the bioluminescence assay, in addition to the pPB-XpreS expression vector, control plasmids (one negative control and one positive control) were used in each host cell system to define the activity of the PB-XpreS promoter.

The negative control is the empty plasmid, that is, not comprising a promoter upstream of the luciferase gene (pGL3B) and represents the background of the luciferase expression.

The positive controls provide a bioluminescence reference value in the four cell systems and are different for each host cell. In particular, the following positive controls were used.
pGL3B-copy provides a bioluminescence reference value in insect cell systems. It is a plasmid comprising the “copy” promoter of retro-transposon origin, widely used for the expression in Drosophila. It has been amplified starting from the pCoBlast plasmid sold by Life Technologies as part of the DES®-Inducible Kit expression system.
pGL3B promoter vector is a plasmid which contains the SV40 promoter of viral origin and provides the bioluminescence reference value in mammalian cell lines. The SV40 promoter is widely used in expression vectors specific to mammalian cell lines. The pGL3-promoter vector plasmid is sold by Promega (catalog number E1761).
pGL3B-cat is a plasmid which comprises the “CAT” promoter, i.e., the promoter of the CAT (chloramphenicol-acetyltransferase) gene and is used because it provides a bioluminescence reference value of bacterial cell systems. The CAT promoter has been amplified starting from the pHSG396 plasmid containing the entire expression cassette for the chloramphenicol acetyl transferase.
pFL39-URA contains the promoter gel gene URA3 of yeast and provides a bioluminescence reference value in yeast cell systems. The URA3 promoter has been amplified starting from the pFL44 plasmid containing the entire expression cassette of the URA3 gene of Saccharomyces cerevisiae.

The cell systems used for the purpose of determining the activity of the promoters in the tested expression vectors are the following:
DH5a™(Invitrogen) (F-Φ80lacZΔM15 Δ(lacZYA-argF) U169 recA1 endA1 hsdR17 (rK−, mK+) phoA supE44 λ-thi-1 gyrA96 relA1), bacterial cell system;
Drosophila S2R+(DGRC) derived from the Schneider's line 2 (Schneider, I. (1972). Cell lines derived from late embryonic stages of Drosophila melanogaster. J. Embryol. exp. Morphol. 27:353-365), insect cell system;
HeK293 human embryonic kidney cells (ATCC), mammalian cell system, specifically humans;
S. cerevisiae BMA64-1A (MAT a ura 3-1 ade 2-1 leu 2-3, 112 his3-11, 15 trp1D can 1-100) yeast cell system.

All cell lines used are in common use in cell biology, basic research as well as on industry level.

The method of inserting plasmid DNA into a cell is different for eukaryotic and prokaryotic cells.

In the case of the eukaryotic cells, the MIRUS TransIt_LT1 (Mims Bio LLC) transfecting agent was used as specified in the supplier's manual. Yeast cells (fungi) were transformed by the lithium chloride method, known per se in the art.

In the case of the prokaryotic cells, the procedure of transformation with calcium chloride, known per se in the art, was used.

Each expression assay has been carried out in triplicate on a suitable number of cells, variable according to the cell type used as experimental model, however in line with the procedures normally used in analogous assays.

The expression level of the luciferase gene, and thus the amount of the protein, has been evaluated based on the amount of light emitted. In fact, the luciferase is able to catalyze a particular chemical reaction during which chemical energy is converted into light energy.

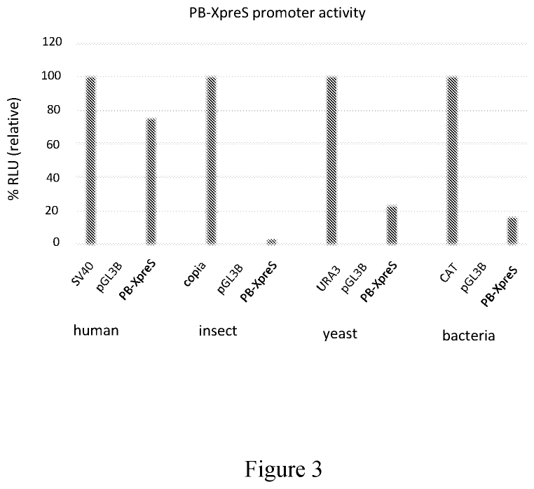

The bioluminescence results are expressed as percentage (%) of relative light units (RLU) and are depicted in FIG. 3.

RLU % detected is directly proportional to the level of synthesized protein and, therefore, to the expression level of the luciferase gene downstream of the various tested promoters. The obtained luminescence results, expressed in Relative Light Units (RLU), were plotted in the graph in FIG. 3, were compared to that of the positive control (arbitrarily set equal to 100%) in each model cell system.

FIG. 3 depicts the results of the bioluminescence assay. The host cell systems are depicted on the abscissa of the graph and are human HeK293 cells (depicted as “human” in FIG. 3), Drosophila melanogaster S2R+cells (depicted as “insect” in FIG. 3), yeast BMA64 cells (depicted as “yeast” in FIG. 3) and bacterial Dh5α cells (depicted as “bacteria” in FIG. 3).

The expression vectors used for the bioluminescence assay are also depicted on the abscissa of the graph. The expression vectors containing different promoters upstream of the luciferase gene are depicted in FIG. 3 as “PB” (vector according to the invention, comprising the sequence of the invention), “SV40” (pGL3B-promoter vector), “copy” (pGL3B-copy vector), “URA3” (pFL39-URA vector) and “CAT” (pGL3B-cat vector). The negative control, that is, the empty plasmid, not containing promoters upstream of the luciferase gene is depicted as “pGL3B” in FIG. 3.

The RLU % is depicted in the ordinate of the graph in FIG. 3.

As may be seen in FIG. 3, the expression vector of the invention (depicted in FIG. 3 as “PB-XpreS”), comprising the PB-XpreS promoter sequence (i.e., SEQ. ID NO. 1), is able to activate the transcription in all the cell systems tested.

The observed expression level of the luciferase gene allowed the promoter of the invention to be classified as a weak promoter. Therefore, advantageously, the promoter of the invention is particularly suitable, for example, to be used for the expression of proteins which, if they are expressed in high amounts, could be toxic for the host cell.

In another test, the gene expressing the resistance to the blasticidin antibiotic inserted between the two terminal sequences (5′-end and 3′-end) of the PiggyBac transposable element, of which the one at the 5′ represents the PB-XpreS sequence according to the invention (SEQ. ID NO. 1), was made to integrate on the chromosomes of human cells.

Transposition assays were then performed by co-expressing PiggyBac transposase with a helper plasmid in human cells (Hapl). The selection with blasticidin (10 ug/mL) applied for 20 days after the transfection denotes the integration on the chromosomes and the stable expression of the resistance gene compared with the negative control.

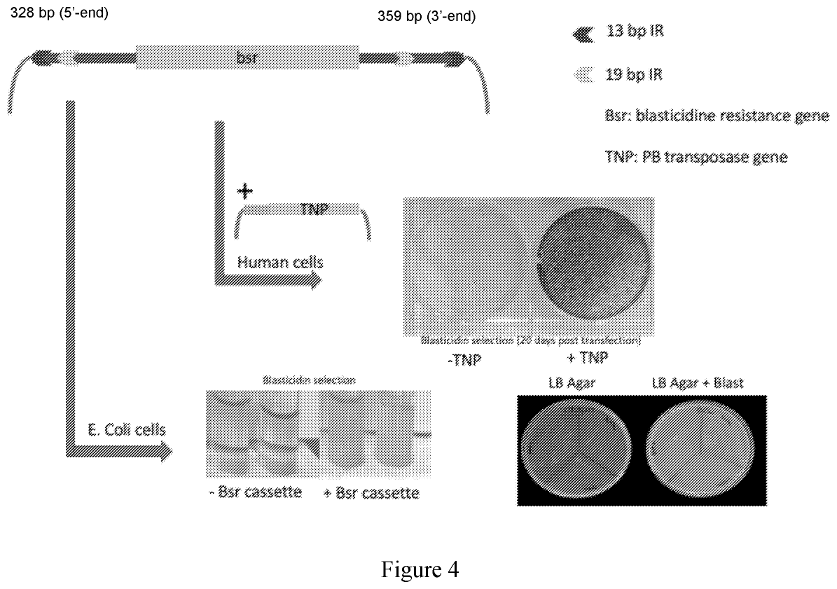

As can be seen in FIG. 4, the co-expression of the gene for blasticidin resistance (denoted as “bsr” in FIG. 4) with the PiggyBac transposase gene (denoted as “TNP” in FIG. 4) allows the selection of the cells expressing the gene for the resistance to the blasticidin antibiotic. In fact, such cells (“+TNP” in FIG. 4) survive exposure to blasticidin, unlike the cells in which the co-expression was not carried out (“−TNP” in FIG. 4). Data were collected 20 days after transfection.

The PiggyBac transposase gene (TNP) allows the transposon (which comprises the PB-XpreS sequence) to be integrated on the chromosomes. Per se, the use of the TNP gene is not necessary for the expression of bsr, which also occurs in un-integrated plasmid form.

Thus, the test demonstrates the applicability of the PB-XpreS promoter of the invention for the stable expression, as well as for the transient expression of target genes.

The same plasmid vector comprising the sequence of the invention (SEQ. ID NO. 1) and the gene for the blasticidin resistance allows the selection in E. coli, by using a concentration of 75 ug/ml blasticidin in the culture medium.

In FIG. 4 the results obtained in E. coli, by using both liquid and solid culture medium (LB Agar), can be observed. In these tests, it was observed that E. coli cells expressing the blasticidin resistance gene (“+Bsr cassette” and “LB Agar+Blast” in FIG. 4) survive exposure to blasticidin, whereas the cells not expressing such resistance (“−Bsr cassette” and “LB Agar” in FIG. 4) do not survive exposure to the antibiotic.

1. A promoter of the transcription in prokaryotic and/or eukaryotic cell systems, which comprises the sequence CCCTAGAAAGATAGTCTGCGTAAAATTGACGCATGCATTCTTGAAATATTGC TCTCTCTTTCTAAATAGCGCGAATCCGTCGCTGTGCATTTAGGACATCTCAG TCGCCGCTTGGAGCTCCCGTGAGGCGTGCTTGTCAATGCGGTAAGTGTCACT GATTTTGAACTATAACGACCGCGTGAGTCAAAATGACGCATGATTATCTTTT ACGTGACTTTTAAGATTTAACTCATACGATAATTATATTGTTATTTCATGTTC TACTTACGTGATAACTTATTATATATATATTTTCTTGTTATAGATATCGTGAC TAATATATAATAAA (SEQ. ID. NO. 1) or a sequence having at least 80% sequence identity with said sequence SEQ ID NO. 1.

2. The promoter according to claim 1, wherein said promoter comprises a sequence having at least 90% sequence identity with said sequence SEQ ID NO. 1.

3. The promoter according to claim 1, wherein said promoter comprises a sequence having at least 90% sequence identity with said sequence SEQ ID NO. 1.

4. A promoter according to claim 1, wherein said promoter has the sequence SEQ. ID. NO. 1.

5. Use of said promoter according to claim 1 for the transcription in prokaryotic and/or eukaryotic cell systems.

6. The use according to claim 5, wherein said prokaryotic systems are bacteria of E. coli species and said eukaryotic systems are selected from mammalian cells, insect cells, yeast cells, reptile cells, amphibian cells and bird cells.

7. An expression vector comprising said promoter according to claim 1.

8. The expression vector according to claim 7, wherein said expression vector is selected from plasmid vector, cosmid, phage, bacteriophage, Yeast Artificial Chromosome (YAC), Bacterial Artificial Chromosome (BAC) or viral vector.

9. The expression vector according to claim 7, further comprising at least one nucleic acid sequence of interest downstream of said promoter according to claim 1.

10. The expression vector according to claim 9, wherein said nucleic acid sequence of interest is a gene sequence.

11. The expression vector according to claim 9, wherein said nucleic acid sequence of interest is upstream of the 3′ terminal sequence of the PiggyBac transposon.

12. The expression vector according to claim 7 for its use in gene therapy.

13. The vector for the use according to claim 12, wherein said gene therapy is the trans-kingdom gene therapy.

14. A method for expressing at least one nucleic acid sequence of interest in a prokaryotic and/or eukaryotic host cell, comprising the steps of:
a) cloning said at least one nucleic acid sequence of interest into an expression vector according to claim 7;
b) introducing said expression vector into said host cell;
c) culturing said host cell to obtain the expression of said nucleic acid sequence of interest.

15. The method for performing the gene therapy with the expression vector according to claim 7, comprising the step of inserting, into specific host cells, said expression vector for the purpose of curing genetic diseases.


SEQUENCE LISTING (3 sequences)

SEQ ID NO: 1 - DNA, 328 bp, synthetic construct
  source 1..328: mol_type = other DNA; note = promoter sequence PB-XpreS derived from Trichoplusia ni
ccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcgtgactaatatataataaa

SEQ ID NO: 2 - DNA, 32 bp, synthetic construct
  source 1..32: mol_type = other DNA; note = primer PB-XpreS_fw (forward)
atcgctcgagccctagaaagatagtctgcgta

SEQ ID NO: 3 - DNA, 32 bp, synthetic construct
  source 1..32: mol_type = other DNA; note = primer PB-XpreS_rev (reverse)
atcgccatggtttattatatattagtcacgat